(19) United States
(12) Patent Application Publication
(10) Pub. No.: US 2022/0257141 A1
(43) Pub. Date: Aug. 18, 2022

(54) METHOD FOR DETERMINING THE FUNCTIONAL RESIDUAL CAPACITY OF A PATIENT'S LUNG AND VENTILATOR FOR CARRYING OUT THE METHOD

(71) Applicant: HAMILTON MEDICAL AG, Bonaduz (CH)

(72) Inventors: Thomas Laubscher, Rhäzüns (CH); Christoph Schranz, Fläsch (CH); Dominik Novotni, Chur (CH); Sascha Reidt, Igis (CH)

(73) Assignee: HAMILTON MEDICAL AG, Bonaduz (CH)

(21) Appl. No.: 17/627,204

(22) PCT Filed: Jul. 15, 2020
(86) PCT No.: PCT/EP2020/069983

§ 371 Date: Jan. 14, 2022

(30) Foreign Application Priority Data
Jul. 18, 2019 (DE) 10 2019 119 575.6

(51) Int. Cl. A61B 5/091; A61M 16/12; A61M 16/20; A61M 16/00

(52) U.S. Cl. CPC A61B 5/091; A61M 16/12; A61M 16/204; A61M 2016/0039; A61M 16/206; A61M 2230/43; A61M 16/0003

(57) ABSTRACT

A method for determining the functional residual capacity of a patient's lung, includes supplying a first inspiratory breathing gas having a first proportion of a metabolically inert gas, supplying a second inspiratory breathing gas having a second proportion of the metabolically inert gas, determining any arising volume difference, which represents a difference in volume between a volume of inspiratory and of expiratory metabolically inert gas for a determination period, determining the functional residual capacity taking into account the volume difference and a proportion difference between a first proportion quantity and a second proportion quantity, which represent the first proportion and the second proportion of the metabolically inert gas, respectively, and determining a base difference, which represents a difference between a tidal volume of inspiratory metabolically inert gas and of expiratory metabolically inert gas.

The present invention relates to a method for ascertaining a functional residual capacity (FRC) of a lung of a patient, comprising the following steps:
supplying a first inspiratory respiratory gas having a first proportion of a metabolically inert gas during a first temporal supply phase,
following the first supply phase: supplying a second inspiratory respiratory gas, differing from the first and having a second proportion of the metabolically inert gas differing from the first, during a second temporal supply phase,
ascertaining a difference in amount occurring during the second supply phase, which represents a difference amount for an ascertainment period between an amount of inspiratory metabolically inert gas and an amount of expiratory metabolically inert gas, the ascertainment period not ending after the second supply phase,
ascertaining the functional residual capacity by taking into account the difference in amount and a difference in proportion between a first proportion quantity, which represents the first proportion of the metabolically inert gas in the first inspiratory working gas, and a second proportion quantity, which represents the second proportion of the metabolically inert gas in the second inspiratory working gas.

Having clarified at the outset that the supply phases are temporal supply phases, the supply phases are designated below without the addition of “temporal”.

A method of this kind is known from Olegård, C. et al.: “Estimation of Functional Residual Capacity at the Bedside Using Standard Monitoring Equipment: A Modified Nitrogen Washout/Washin Technique Requiring a Small Change of the Inspired Oxygen Fraction”, in International Anesthesia Research Society: “Anesthesia & Analgesia”, 2005 (101), pages 206-212. This method is referred to in the technical literature and in the present application as the “method according to Olegård”.

Such methods for ascertaining the functional residual capacity, or FRC, are generally known as wash-out methods, in contrast to likewise existing dilution methods and to the known body plethysmography, since these methods are based on an observation of washing the metabolically inert gas out of the lung of the patient following a change in the composition of inspiratory respiratory gas.

Wash-out methods are likewise known from Wauer, H. J. et al.: “FRC-Messung bei beatmeten Intensivpatienten—Eine Standortbestimmung” [“FRC measurement in ventilated intensive care patients—and assessment”], in Springer Verlag: “Der Anaesthesist” [“The Anesthesiologist”], 1998 (47), pages 844-855.

A further wash-out method and a device for this purpose are known from U.S. Pat. No. 7,530,353 B2.

The ascertainment of an FRC by wash-out methods is based on ascertaining the amount of washed out metabolically inert gas during a wash-out as well as on ascertaining the difference in proportions of metabolically inert gas in the inspiratory respiratory gas prior to and during the wash-out process. In connection with the present application, a wash-out process is understood as at least the time span that extends from a point in time at the start of the second supply phase to the point in time in the second supply phase, at which the lung of the patient and thus the expiratory respiratory gas flowing out of it reveals again an essentially constant proportion of the metabolically inert gas over multiple breaths during a ventilation with the second inspiratory respiratory gas. The wash-out process and thus the ascertainment period may be shorter than the second supply phase. It is not longer than the second supply phase, however.

A condition for the start of the second supply phase is that the expiratory respiratory gas in the first supply phase has an essentially constant proportion of the metabolically inert gas. Otherwise, the wash-out method may indeed be carried out, but it will result in an inaccurate or false result.

The above-mentioned publications of the related art differ essentially in the calculation of the amount of washed out metabolically inert gas. This amount of washed out metabolically inert gas is represented in the present application by the difference in amount between an amount of inspiratory and an amount of expiratory metabolically inert gas. While in the method according to Olegård, for each breath, a tidal difference in quantity is formed between the amounts of metabolically inert gas in the inspiratory and in the expiratory respiratory gas and is summed up over the duration of the wash-out process, Wauer et al. as well as the U.S. Pat. No. 7,530,353 B2 ascertain the amount of washed out metabolically inert gas by ascertaining the tidal amount of metabolically inert gas in the expiratory respiratory gas immediately prior to the start of the second supply phase as a base amount and by summing up tidal differential values that are formed by subtracting the base amount from the respective tidal amount of metabolically inert gas in the expiratory respiratory gas over the breaths of the wash-out process.

A certain advantage of the last-mentioned calculation method is that its algorithm is independent of the inspiratory respiratory gas. It is based on the assumption that any difference in the tidal amounts of metabolically inert gas exhaled per breath compared to the exhaled tidal amount prior to the start of the second supply phase diminishes solely through the wash-out of metabolically inert gas still present in the lung of the patient at the start of the second supply phase. This assumption, however, is justified at best under ideal laboratory conditions, in which for example inspiratory respiratory gas can be supplied to the patient without leakage. If the actual supply conditions deviate from the ideal laboratory conditions, however, which is the case even under favorable conditions in the laboratory, the inaccuracy of the last-mentioned calculation method increases.

The method according to Olegård, on the other hand, is based on the assumption that the difference between the inhaled and the exhaled tidal amounts of metabolically inert gas is based solely on a wash-out of metabolically inert gas still present in the lung of the patient at the start of the second supply phase. This assumption is in principle reasonable since the term “metabolically inert” indicates precisely that a substance in the body of the patient labeled in this manner is not metabolized, so that initially there appears to be no reason why the expiratory and the inspiratory respiratory gas should not contain the same amounts of metabolically inert gas. Since the method according to Olegård also takes the inspiratory respiratory gas into account, it theoretically provides more accurate results than the previously discussed calculation method. In the cited article, however, Olegård describes the problems in the detection of the amount of metabolically inert gas in the inspiratory respiratory gas, which is why in the method according to Olegård, the values of the inspiratory respiratory gas are not detected by measurement, but are rather derived on the basis of a Haldane transformation from the values of the expiratory respiratory gas which alone are detected by measurement. Nevertheless, the method according to Olegård provides more accurate results than the other methods mentioned above.

The objective of the present invention is to allow for the ascertainment of the functional residual capacity of a lung of a patient in the normal course of a clinical day, i.e., without a special laboratory environment or laboratory conditions. In particular, the ascertainment of the functional residual capacity is to be possible using a ventilator during a ventilation operation for the at least partially artificial respiration of a patient.

Relevant experiments have shown that when using a wash-out method in everyday clinical situations, the tidal difference in amount between the proportion of metabolically inert gas in the inspiratory respiratory gas and the proportion of metabolically inert gas in the expiratory respiratory gas does not become 0 even long after the start of the second supply phase, but that it rather approaches or takes on an offset value. According to current, still incomplete knowledge, several causes are regarded as responsible for this fact: on the one hand, leakages exist in the ventilation line systems by which inspiratory respiratory gas is supplied to the patients and expiratory respiratory gas is conducted away from the patient, which may change the respiratory gas in its composition. On the other hand, measuring errors may occur in the flow measurement of the respiratory gas flow, for example due to asymmetrical incident flow on a flow resistor of a differential pressure flow sensor and/or due to asymmetrical moistening and/or droplet formation in the differential pressure flow sensor etc.

The assumptions tend in the direction that on the one hand the ventilator as such and on the other hand the ventilation situation produced by the respective ventilator may respectively have an influence of error on the ascertainment of the FRC.

It is therefore the objective of the present invention to improve the method mentioned at the outset in such a way that it may be carried out with sufficiently high accuracy of the result even in a clinical environment lacking laboratory quality.

According to the invention, this objective is achieved in the method mentioned at the outset in that the method comprises the following further steps:
ascertaining a base difference, which represents a difference between a tidal amount of inspiratory metabolically inert gas and a tidal amount of expiratory metabolically inert gas in the first and/or the second supply phase,

the ascertainment of the functional residual capacity occurring on the basis of a corrected difference in amount and the difference in proportion, the corrected difference in amount being formed by taking into account the base difference when ascertaining the difference in amount.

The described base difference is a quantity specific to a single breath and represents a measure for the above-described offset value, which may exist on a ventilator, by which the method of the invention is carried out, even long after the start of the second supply phase. Thus, the neither known nor predictable influence of the ventilator and/or the influence of the respectively prevailing ventilation situation may be quantified and taken into account in the calculation of the FRC. The value of the FRC ascertained as a result of the method may thus be obtained reliably and with very good accuracy even in environments, in which the environmental conditions change and/or in which the environmental conditions are partially unknown.

The ascertainment period may be the entire second supply phase or may be a temporally shorter period than the second supply phase. For example, the ascertainment period may end when differential values, which represent differences in tidal amounts between the tidal amount of metabolically inert gas in the inspiratory respiratory gas and the tidal amount of metabolically inert gas in the expiratory respiratory gas, differ for successive breaths according to amount by less than a predetermined difference threshold value. Then a state has been reached for the second supply phase, in which a sufficient equilibrium, determined by the difference threshold value, between the amounts of metabolically inert gas in the inspiratory and in the expiratory respiratory gas is reached. The ascertainment period then corresponds effectively to the duration of a wash-out process or also of a wash-in process. A wash-in process corresponds to a wash-out process with the sole difference that in the wash-out process the proportion of metabolically inert gas is higher in the first inspiratory respiratory gas than in the second inspiratory respiratory gas, while precisely the opposite is the case in a wash-in process.

The ascertainment period preferably starts at the same time as the second supply phase.

In order to allow for an ascertainment of the FRC that is as accurate as possible, the transition from the first to the second supply phase should be as short as possible, if possible shorter than one breath. The transition from the first to the second supply phase occurs particularly preferably during an expiration phase of the patient, so that during the inspiration phase immediately preceding the expiration phase, the patient still receives the first inspiratory respiratory gas and that during the inspiration phase immediately following the expiration phase, the patient already receives the second inspiratory respiratory gas.

The difference in tidal amounts of an nth breath following the start of the ith supply phase, in which it occurred, may be represented formulaically as:

^{iZp}Δv(n)_{miG}^{tid}=ᵢₙ^{iZp}v(n)_{miG}^{tid}−ₑₓ^{iZp}v(n)_{miG}^{tid} (eq. 1)

where “tid” stands for “tidal”, “miG” for metabolically inert gas and “iZp” indicates the supply phase, where i=1 for the first supply phase and i=2 for the second supply phase, ^{iZp}Δv(n)_{miG}^{tid} furthermore designating the difference in tidal amounts of the nth breath of the ith supply phase, ᵢₙ^{iZp}v(n)_{miG}^{tid} designating the tidal amount of metabolically inert gas in the inspiratory respiratory gas in the nth breath of the ith supply phase, and ₑₓ^{iZp}v(n)_{miG}^{tid} designating the tidal amount of metabolically inert gas in the expiratory respiratory gas in the nth breath of the ith supply phase. For each supply phase, n increases incrementally starting anew from 1, according to the present application.

There exist several possibilities for ascertaining the tidal amounts ᵢₙ^{iZp}v(n)_{miG}^{tid} of inspiratory metabolically inert gas and ₑₓ^{iZp}v(n)_{miG}^{tid} expiratory metabolically inert gas. On the one hand, it is possible to ascertain these values, as it is known in the related art from the Olegård publication cited above.

According to the formula by Bohr that is known per se, Olegård ascertains a tidal amount of expiratory alveolar respiratory gas from an expiratory amount of CO₂ ascertained over a predetermined period, an endexpiratory or endtidal CO₂ proportion value associated with this period, which respectively indicates the endexpiratory or endtidal proportion of CO₂ in the breaths of the predetermined period, and the number of breaths in the predetermined period. This is shown formulaically in the following equation 1a:

$${}_{ex}^{iZp} v {( n )}_{alv}^{tid} = \frac{{}_{ex}^{iZp} {v}_{CO 2}^{mean}}{{}_{ee}^{iZp} {a}_{CO 2}^{mean} \cdot k} \qquad ( eq . 1 a )$$

with ₑₓ^{iZp}v(n)ₐₗᵥ^{tid} as the tidal alveolar expiratory respiratory gas amount of the nth breath, with ₑₓ^{iZp}v_{CO2}^{mean} as the amount of CO₂ in the expiratory respiratory gas averaged over k breaths, and with ₑₓ^{iZp}a_{CO2}^{mean} as the proportion of CO₂ in the endexpiratory respiratory gas averaged over k breaths. If the tidal expiratory respiratory gas amount is known and the tidal inspiratory respiratory gas amount is known, the tidal alveolar inspiratory respiratory gas amount ₑₓ^{iZp}v(n)ₐₗᵥ^{tid} may be determined in a manner known per se from the tidal alveolar expiratory respiratory gas amount. It is true both for the inspiratory as well as for the expiratory aspect that a tidal respiratory gas amount is the sum of the tidal dead space amount of the respiratory system and the tidal alveolar amount. The expiratory tidal amount of metabolically inert gas ₑₓ^{iZp}v(n)_{miG}^{tid} of the nth breath may then be ascertained from the tidal alveolar expiratory respiratory gas amount ₑₓ^{iZp}v(n)ₐₗᵥ^{tid} and the proportion of metabolically inert gas in the endexpiratory respiratory gas ₑₓ^{iZp}v(n)_{miG}^{tid} of the nth breath according to the following equation 1 b:

ₑₓ^{iZp}v(n)_{miG}^{tid}=ₑₓ^{iZp}v(n)ₐₗᵥ^{tid}·ₑₑ^{iZp}a(n)_{miG}^{tid} (eq. 1b)

Instead of the tidally calculated proportion ₑₑ^{iZp}a(n)_{miG}^{tid}, it is also possible to use a proportion of metabolically inert gas averaged over multiple breaths.

In an analogous manner, the tidal amount of metabolically inert gas in the inspiratory respiratory gas ᵢₙ^{iZp}v(n)_{miG}^{tid} may be ascertained on the basis of the tidal alveolar inspiratory respiratory gas amount ᵢₙ^{iZp}v(n)ₐₗᵥ^{tid} and the tidally ascertained proportion of metabolically inert gas in the inspiratory respiratory gas ᵢₙ^{iZp}a(n)_{miG}^{tid} of the nth breath in accordance with equation 1c:

ᵢₙ^{iZp}v(n)_{miG}^{tid}=ᵢₙ^{iZp}v(n)ₐₗᵥ^{tid}·ᵢₙ^{iZP}a(n)_{miG}^{tid} (eq. 1c)

Again, instead of a tidally ascertained proportion, it is possible to use a proportion value averaged over multiple breaths. The tidally ascertained proportion values are normally values averaged over a respective tidal partial respiratory process: expiration and inspiration.

Endtidal or endexpiratory values are preferred for this purpose, since the endtidal or endexpiratory respiratory gas observable toward the end of a breath or an expiration process originates with certainty from the metabolizing area of the lung and certainly not from a dead space of the respiratory system.

It was discovered, however, that for patients having an obstructive-pathological lung for example, equations 1a and 1b do not provide optimal values due to their focus on the endtidal phase of a breath. It has therefore proven advantageous to use values that were ascertained in a middle period of the expiration instead of values ascertained in endexpiratory fashion. Equation 1a then transitions into the following equation 1d:

$${}_{ex}^{iZp} v {( n )}_{alv}^{tid} = \frac{{}_{ex}^{iZp} {v}_{CO 2}^{mean}}{{}_{zent}^{iZp} {a}_{CO 2}^{mean} \cdot k} \qquad ( eq . 1 d )$$

where _{zent}^{iZp}a(n)_{CO2}^{mean} is a proportion value of CO₂ in the expiratory respiratory gas averaged over k breaths, whose individual values were respectively determined in a central period of the respective expiration phase, that is, at a point in time that is closer to the temporal middle of the expiration phase than to the start or the end of the expiration phase. The detection time preferably lies within a period that is no longer than 20% of the duration of the expiration phase and that extends symmetrically around the temporal middle of the expiration phase. Particularly preferably, at least one proportion value is, or preferably the respective proportion values are ascertained in the temporal middle of the respective expiration phase.

The same applies also for ascertaining the proportion of metabolically inert gas in the expiratory respiratory gas. Equation 1b then turns into the following equation 1e:

ₑₓ^{iZp}v(n)_{miG}^{tid}=ₑₓ^{iZp}a(n)ₐₗᵥ^{tid}·_{zent}^{iZp}a(n)_{miG}^{tid} (eq. 1e)

where _{zent}^{iZp}a(n)_{miG}^{tid} is a proportion of metabolically inert gas in the expiratory respiratory gas that is respectively tidally determined in a central period of the respective expiration phase of the nth breath. With regard to the preferred point in time for determining the proportion value, what was said above about the CO₂ proportion value applies here as well.

The tidal inspiratory amount of metabolically inert gas may be calculated without change according to equation 1c.

Surprisingly, an even more accurate result of the difference in tidal amounts may be obtained, if the individual tidal amounts of the right side of equation 1 are obtained on the basis of an averaged tidal inspiratory respiratory gas amount, multiplied by the mean proportion of metabolically inert gas of the respective inspiration phase, as well as on the basis of an averaged tidal expiratory respiratory gas amount, multiplied by the mean proportion of metabolically inert gas of the respective expiration phase.

A mean tidal inspiratory respiratory gas amount ᵢₙ^{iZp}v^{mean} may be obtained by averaging over multiple tidal inspiratory respiratory gas amounts. Since the tidal inspiratory as well as the tidal expiratory respiratory gas amounts are normally not influenced by changes in the respiratory gas composition, the averaging may also be performed across the boundary between two supply phases. The tidal inspiratory amount of metabolically inert gas of the nth breath may then be ascertained according to the following equation 1f instead of equation 1c:

ᵢₙ^{iZp}v(n)_{miG}^{tid}=ᵢₙ^{iZp}v^{mean}·ᵢₙ^{iZp}a(n)_{miG}^{tid} (eq. 1f)

where ᵢₙ^{iZp}a(n)_{miG}^{tid} is again the tidally ascertained proportion of metabolically inert gas in the inspiratory respiratory gas of the nth breath.

Accordingly, the tidal expiratory amount of metabolically inert gas may be ascertained according to the following equation 1 g:

ₑₓ^{iZp}v(n)_{miG}^{tid}=ₑₓ^{iZp}v^{mean}·ₑₓ^{iZp}a(n)_{miG}^{tid} (eq. 1g)

where ₑₓ^{iZp}a(n)_{miG}^{tid} is again the tidally ascertained proportion of metabolically inert gas in the expiratory respiratory gas of the nth breath, and where ₑₓ^{iZp}v^{mean} is a mean tidal expiratory respiratory gas amount ascertained by averaging over multiple tidal respiratory gas amounts. What was said above regarding the mean tidal inspiratory respiratory gas amount applies accordingly mutatis mutandis to the mean tidal expiratory respiratory gas amount.

A differential value may be the difference in the tidal amounts occurring in a breath itself. Every differential value then represents the breath of its difference in the tidal amounts. In order to smooth out unavoidable fluctuations of the differences in tidal amounts of individual breaths, the differential values may be average values, which take into account differences in tidal amounts of a plurality of breaths. The differences in tidal amounts may be moving averages for example. For better comparability, each moving average takes into account the same number of individual values. The average values may comprise or be—preferably—arithmetic average values. Alternatively, they may also comprise or be geometric average values. A differential value D(n) calculated over k breaths as a moving arithmetic average and representing the nth breath in the ith supply phase may therefore be represented formulaically as

$$D ( n ) = \frac{1}{( k + 1 )} . {\sum }_{x = n - k}^{n} {}^{iZp} Δ {v ( x )}_{m i G}^{t i d} \qquad ( eq . 2 )$$

where ^{iZp}Δv(n)_{miG}^{tid} may be calculated in accordance with equation 1.

Since with the progressive duration of the second supply phase the differences in tidal amounts approach the offset value mentioned at the outset, the average values may have a weighting, preferably a weighting, in which the differences in tidal amounts of breaths that are closer in time to the current breath, preferably including the current breath, are more heavily weighted than the differences in tidal amounts of breaths that are further away in time from the current breath. When using moving averages, an average value counts as representing the particular breath for which the temporally last individual value was ascertained, which is taken into account in the calculation of the moving average. The comparison of a difference of differential values with the predetermined difference threshold value may comprise a subtraction of a first differential value, which represents a specific breath, from a second differential value, which represents a breath following immediately upon the predetermined breath. The immediately following breath is preferably the respective current breath.

Here it shall be pointed out expressly that the presently described method is not necessarily only a wash-out method used for describing the related art, but may also be a wash-in method in a reversal of the principle of a wash-out method. The second proportion of metabolically inert gas in the second inspiratory respiratory gas is therefore not necessarily lower than the corresponding first proportion in the first inspiratory respiratory gas. It may also be higher than the first proportion. If one defines the difference in amount mentioned at the outset, which occurs during the ascertainment period in the second supply phase, as

^{2Zp}Δv_{miG}=ᵢₙ^{2Zp}v_{miG}−ₑₓ^{2Zp}v_{miG} (eq. 3)

with ^{2Zp}Δv_{miG} as the difference in amount occurring during the ascertainment period in the second supply phase between the amount of metabolically inert gas in the inspiratory respiratory gas (this is also referred to as the amount of inspiratory metabolically inert gas in the present application) and the amount of metabolically inert gas in the expiratory respiratory gas (this is also referred to as the amount of expiratory metabolically inert gas in the present application), with ^{2Zp}v_{miG} as the amount of inspiratory metabolically inert gas administered in the second supply phase and with ₑₓ^{2Zp}v_{miG} as the amount of expiratory metabolically inert gas exhaled in the second supply phase, then the difference in amount occurring during the second supply phase is negative for a wash-out method and positive for a wash-in method. For in sum more metabolically inert gas is exhaled than inhaled in the wash-out method. In the wash-in method the reverse is the case.

Fundamentally, what was said above regarding the differential values also applies to the base difference. It is possible to form the base difference from the difference in tidal amount solely of an individual breath, preferably of a breath toward the end of the first or of the second supply phase, particularly preferably of the second supply phase. This is not preferred, however, due to the possible fluctuations of the individual tidal measurement values for determining the difference in tidal amounts from breath to breath. The base difference therefore comprises preferably at least one average value from a plurality of differences in tidal amounts between respectively a tidal amount of inspiratory metabolically inert gas and a tidal amount of expiratory metabolically inert gas for a plurality of breaths in the first and/or the second supply phase. Again, the relevant average value may comprise or be an arithmetic average value. Alternatively, the average value may comprise or be a geometric average value. This allows for the smoothing out of the fluctuation of detection values that was addressed above. The average value may be a moving average or may be an average value, which is ascertained on the basis of a predetermined number of breaths following the elapse of a number of breaths since the start of the second supply phase.

The base difference is preferably an average value from a plurality of differences in tidal amounts of breaths, which occurred in the second supply phase, since the offset values of the differences in tidal amounts of the first supply phase and of the second supply phase may differ and normally actually differ in practice. The base difference then represents the offset value of the second supply phase, which promises a higher accuracy of the ascertained FRC than a base difference that represents the offset value of the first supply phase. Since normally, however, the offset values of the first and of the second supply phases have the same sign, the use of a base difference on the basis of breaths of the first supply phase, which consequently represents the offset value of the first supply phase, still provides a better accuracy than if no base difference were taken into account.

A base difference ^{iZp}B calculated as the arithmetic average value for the ith supply phase may therefore be written in the manner of equation 2 for the differential value as:

$${}^{iZp} B = \frac{1}{( m + 1 )} \cdot {\sum }_{x = {n}_{0} - m}^{{n}_{0}} {}^{iZp} Δ {v ( x )}_{m i G}^{t i d} \qquad ( eq . 4 )$$

where no is particularly preferably the number of the last breath of the ascertainment period or of the ith supply phase (Zp), and where m is the number of individual values of differences in tidal amounts, which are taken into account for averaging the base difference. Generally, it is preferred that the magnitude of the interval between no and the number of the last breath of the ascertainment period or of the ith supply phase is smaller than n₀−m−1. The latter is the interval of the number n₀−m of the first breath, which is taken into account in the formation of the base difference, from the first breath of the ascertainment period or the ith supply phase. It is preferred that the first breath of the ascertainment period is identical with the first breath of the second supply phase.

Since the desired state of equilibrium, in which differences in tidal amounts between respectively a tidal amount of inspiratory metabolically inert gas and a tidal amount of expiratory metabolically inert gas change as little as possible over multiple breaths, sets in with increasing distance from the start of a supply phase, it is preferred for the purpose of achieving a highest possible accuracy in the ascertainment of an FRC that the base difference is an average value from a plurality of differences in tidal amounts between respectively a tidal amount of inspiratory metabolically inert gas and a tidal amount of expiratory metabolically inert gas for a plurality of breaths in a temporal start detection segment in the first supply phase, the start detection segment being closer to the start of the second supply phase than to the start of the first supply phase. This thus applies to the case in which the base difference is based on detection values of breaths of the first supply phase.

Additionally or alternatively, for the same reason of obtaining a highest possible accuracy of the ascertainment result of the FRC, there may be a provision for the base difference to comprise an average value from a plurality of differences in tidal amounts between respectively a tidal amount of inspiratory metabolically inert gas and a tidal amount of expiratory metabolically inert gas for a plurality of breaths in a temporal end detection segment in the second supply phase, the end detection segment being closer to the end of the second supply phase than to its start. Since the ascertainment period is situated in the second supply phase, the ascertainment of the base difference from differences in tidal amounts of breaths of the second supply phase is preferred for the reasons, already described above, of achieving a higher accuracy in the ascertainment of the FRC.

Alternatively or additionally, it is possible for the ascertainment of the base difference to occur only once a difference in tidal amount between a tidal amount of inspiratory metabolically inert gas and a tidal amount of expiratory metabolically inert gas falls below a base threshold value. This ensures that the base difference occurs only on the basis of breaths with differences in tidal amounts, in which the lung of the patient is largely washed out or washed in, depending on the method used, and that consequently the exchange of gas is so close to the state of equilibrium mentioned above that it is possible to ascertain a base difference that is expressive with respect to the offset value setting in with the utilized ventilator and/or in the respective ventilation situation. The selection of the magnitude of the base threshold value determines how close the lung of the patient is to the state of equilibrium at the start of an ascertainment of the base difference. Normally, in an expedient selection of the base threshold value, the above condition will also be fulfilled, that the breaths, on the detection values of which the ascertainment of the base difference is based, will for the most part or preferably completely be closer to the end of the supply phase, in which the breaths occur, than to its start.

The accuracy of the ascertainment of an FRC of a patient may be increased further, however, by considering and calculating the base difference in a more detailed and differentiated manner.

Without going into the details of the physiological-physical relationships, in experiments, the offset value has repeatably shown a dependency on the proportion of metabolically inert gas in the respiratory gas. For this reason, the FRC ascertained using the presently proposed method may be even closer to the FRC ascertained for verification using known recognized ascertainment methods, if the base difference is ascertained at least in time sections depending on the proportion of metabolically inert gas in the respiratory gas and used for ascertaining the FRC.

In a development of the present FRC ascertainment method that is preferred on account of the accuracy of the FRC ascertainment achieved thereby, this may be done in that at least for a plurality of breaths a tidal base difference is ascertained, which is a function of the proportion of metabolically inert gas in the respiratory gas during the entire respective breath. Preferably, the tidal base difference is ascertained as a function of a tidal proportion of metabolically inert gas in the respiratory gas averaged over the respective breath, the averaged tidal proportion preferably taking into account both the proportion in the inspiratory respiratory gas as well as in the expiratory respiratory gas, in order to model the wash-out or the wash-in process.

More precisely, the tidal base difference may be a relationship of a function of a tidal proportion of metabolically inert gas in the respiratory gas averaged over the entire respective breath to the difference in proportion mentioned at the outset. The function may be for example the proportion of metabolically inert gas in the respiratory gas averaged over the respective breath itself, or, preferably, it may be the difference between the proportion of metabolically inert gas in the respiratory gas averaged over the respective breath and the first proportion quantity.

Preferably, the tidal base difference during the second supply phase has values, which according to amount do not exceed an averaged, preferably in accordance with the above equation 4, base difference ^{2Zp}B for the second supply phase. Since prior to the second supply phase, that is, in the first supply phase, an averaged, preferably again in accordance with the above equation 4, base difference ^{1Zp}B is relevant for the first supply phase, the tidal base difference during the second supply phase particularly preferably assumes only values, which according to amount lie between ^{1Zp}B and ^{2Zp}B, the respective limits included.

Usually, tidal values of only the second supply phase suffice for ascertaining the FRC. It consequently suffices to calculate a tidal base difference ^{2Zp}B^{tid}(n) only for breaths of the second supply phase.

With ᵢₙ^{1Zp}A_{miG} as the first proportion quantity and with ᵢₙ^{2Zp}A_{miG} as the second proportion quantity, the tidal base difference of the nth breath of the second supply phase may be formulaically represented as:

$${}^{2 Zp} {B}^{tid} ( n ) ∼ \frac{f ( {}^{2 Zp} a {( n )}_{miG}^{tid} )}{{}_{in}^{2 Zp} {A}_{miG} - {}_{in}^{1 Zp} {A}_{miG}} \qquad ( eq . 5 )$$

with f as function and with ^{2Zp}a(n)_{miG}^{tid} as the tidal proportion of the metabolically inert gas, averaged over the nth breath of the second supply phase, of the entire tidal respiratory gas. The tidal proportion is therefore preferably averaged over the inspiratory phase and over the expiratory phase. The method mentioned above as particularly preferred for calculating the tidal base difference of the nth breath in the second supply phase is represented formulaically in the following equation 6:

$${}^{2 Zp} {B}^{tid} ( n ) = {}^{1 Zp} B + ( {}^{2 Zp} B - {}^{1 Zp} B ) ( \frac{{2 Zp}^{} {a ( n )}_{miG}^{tid} - {}_{in}^{1 Zp} {A}_{miG}}{{}_{in}^{2 Zp} {A}_{miG} - {}_{in}^{1 Zp} {A}_{miG}} ) \qquad ( eq . 6 )$$

where preferably ^{1Zp}B and ^{2Zp}B are preferably calculated in accordance with the above equation 4 and where ᵢₙ^{2Zp}A−ᵢₙ^{1Zp}A in the denominator corresponds to −ΔA from the equation 9 below and is therefore preferably calculated by using the equation 9 explained in more detail below as (−1·ΔA).

Preferably, the corrected difference in amount is formed from a sum of corrected differences in tidal amounts. In the preferred execution of the present method by a ventilator during a ventilation operation, values of the respiratory gas are detected in any case with every breath, in particular both of the inspiratory as well as of the expiratory respiratory gas. It is therefore advantageous to utilize the tidal detection values, that is, the detection values specific to the individual breath, which exist in any case, also for ascertaining the FRC. In that case, the corrected difference in amount can correspond to a sum of corrected differences in tidal amounts over a number of breaths in the ascertainment period, a corrected difference in the tidal amount between a tidal amount of inspiratory metabolically inert gas and a tidal amount of expiratory metabolically inert gas being formed for every breath from a difference of a difference in the tidal amount of this breath and a base difference associated with the breath, the difference in the tidal amount being formed for every breath by the difference between a tidal amount of inspiratory metabolically inert gas and a tidal amount of expiratory metabolically inert gas of this breath. In a preferred specific embodiment of the method, the corrected difference in amount may be represented formulaically as follows:

ₖₒᵣᵣ^{2Zp}Δv_{miG}=Σ_{x=c}_{₀}ⁿ^{₀}(^{2Zp}Δv(x)_{miG}^{tid}−^{iZp}B) (eq. 7a)

where ₖₒᵣᵣ^{2Zp}Δv_{miG} is the corrected difference in amount and ^{2Zp}Δv_{miG}^{tid} is the difference in the tidal amount of the xth breath in the ascertainment period situated entirely within the second supply phase. n₀ is the number of the last breath of the ascertainment period, c₀ is the number of the first breath of the ascertainment period within the second supply phase. If, which is preferred, c₀=1 is chosen, then the first breath of the ascertainment period is also the first breath of the second supply phase. A base difference ^{2Zp}B ascertained in accordance with the above equation 4 from breaths of the second supply phase is preferably used as base difference ^{iZp}B. As explained above, however, it is also possible to use a base difference ^{1Zp}B ascertained in accordance with equation 4 from breaths of the first supply phase, which is less preferred, however, due to the lower accuracy of the FRC ascertainable from this.

Due to the particularly high accuracy in the FRC ascertainment, the corrected difference in amount is preferably calculated using the following modified equation 7b:

ₖₒᵣᵣ^{2Zp}Δv_{miG}=Σ_{x=c}_{₀}ⁿ^{₀}(^{2Zp}Δv(x)_{miG}^{tid}−^{2Zp}B^{tid}(x)) (eq. 7b)

where ^{2Zp}B^{tid}(x) is preferably ascertained in accordance with equation 6.

The first and/or the second proportion of metabolically inert gas in the first or in the second inspiratory respiratory gas may also fluctuate according to amount during the first or during the second supply duration. In principle, as the first proportion quantity, a first proportion detected at a specific time may be used as the sole detection value. The same applies respectively to the second proportion quantity. In order to be able to smooth out possibly occurring fluctuations according to amount in the first and/or in the second proportion, however, it is preferred if the first proportion quantity comprises or is an average value, formed over a plurality of breaths in the first supply phase, of the first proportion of the metabolically inert gas in the first inspiratory working gas.

Additionally or alternatively, for the same reason, the second proportion quantity may comprise or be an average value, formed over a plurality of breaths in the second supply phase, of the second proportion of the metabolically inert gas in the second inspiratory working gas.

Again, the average value may comprise or be an arithmetic average value. The average value may likewise comprise or be a geometric average value. The average value may be weighted.

Since the first proportion of metabolically inert gas in the first inspiratory respiratory gas ideally does not change during the first supply phase, and since ideally the same applies for the second proportion during the second supply phase, it is in the first approximation of secondary importance, on which breaths of the first and of the second supply phase, respectively, the first proportion quantity and the second proportion quantity are determined. If fluctuations according to amount occur, however, in the first and/or in the second proportion during the first and, respectively, during the second supply phase, the ascertainment result of the FRC may be obtained with high accuracy in spite of these fluctuation in that the plurality of breaths, over which the first proportion quantity is ascertained as average value, is closer to the start of the second supply phase than to the start of the first supply phase. For the closer temporally the first inspiratory respiratory gas is considered to the start of the second supply phase, the greater is the influence of the considered respiratory gas on the wash-out or wash-in of metabolically inert gas during the second supply phase.

Additionally or alternatively, for the purpose of increasing the accuracy of the ascertainment result of the FRC, it is advantageous if the plurality of breaths, over which the second proportion quantity is ascertained as an average value, is situated closer to the end of the ascertainment period or of the second supply phase than to their start. The reason for this is that the second proportion of metabolically inert gas in the second inspiratory respiratory gas has an influence on the offset value, described at the outset, in the second supply phase that is all the more pronounced, the closer it is situated to the end of the ascertainment period. If the ascertainment period has ended, subsequent breaths have no more influence on the ascertainment of the FRC. Nevertheless, for simplifying the control, it is possible to chose the end of the second supply phase instead of the end of the ascertainment period, so that the second proportion quantity may be determined independently of the end of the ascertainment period.

A preferred proportion quantity calculated as an arithmetic average value may be formulaically represented as follows:

$${}_{in}^{iZp} {A}_{miG} = \frac{1}{( q + 1 )} \cdot {\sum }_{x = p - q}^{p} {}_{in}^{iZp} a {( x )}_{miG}^{tid} \qquad ( eq . 8 )$$

where ^{iZp}A_{miG} is for i=1 the first proportion quantity and for i=2 the second proportion quantity, where ^{iZp}a(n)_{miG}^{tid} is the proportion of metabolically inert gas in the inspiratory respiratory gas of the xth breath in the ith supply phase and where p and q are positive integer constants with p>q, which indicate the number of values, respectively detected during another breath, over which the ith proportion quantity is calculated by averaging. p and q as numbers of a breath are preferably chosen in such a way that for the first supply phase the interval between the last breath and the pth breath of the first supply phase is greater than the interval of the qth breath from the first breath of the first supply phase.

Equation 8 is provided for calculating the proportion of metabolically inert gas in the inspiratory respiratory gas. Using equation 8, it is in principle possible to calculate all proportions of individual gas components of a gas mixture, both in the inspiratory as well as in the expiratory respiratory gas.

Since with increasing temporal distance from the start of the second supply phase the values of the proportions of metabolically inert gas in the inspiratory and in the expiratory respiratory gas assimilate to each other, —for with increasing distance from the start of the second supply phase previously washed in metabolically inert gas is washed out and previously washed out metabolically inert gas is washed in—the proportion of metabolically inert gas in the expiratory respiratory gas ascertained with sufficient distance from the start of the second supply phase also represents the proportion of metabolically inert gas in the inspiratory respiratory gas and may therefore be used as the second proportion quantity. The same applies to the use of a proportion of the metabolically inert gas in the expiratory respiratory gas ascertained with sufficient distance from the start of the first supply phase for determining the first proportion quantity.

For the second supply phase, p and q are preferably chosen in such a way that the distance between the last breath and the pth breath of the second supply phase or of the ascertainment period is greater than the distance of the qth breath of the second supply phase from the first breath of the second supply phase or of the ascertainment period.

For values of p and q chosen in this way, equation 8 as modified equation 8a also applies for the summation of proportions of metabolically inert gas in the expiratory respiratory gas or in the respiratory gas as a whole:

$${}_{in}^{iZp} {A}_{miG} = \frac{1}{( q + 1 )} \cdot {\sum }_{x = p - q}^{p} {}_{ex}^{iZp} a {( x )}_{miG}^{tid} \approx \frac{1}{( q + 1 )} \cdot {\sum }_{x = p - q}^{p} {}^{iZp} a {( x )}_{miG}^{tid} \qquad ( eq . 8 a )$$

Analogous to the first inspiratory respiratory gas, in the present application, respiratory gas exhaled during the first supply phase is also referred to as first expiratory respiratory gas and respiratory gas exhaled during the second supply phase is also referred to as second expiratory respiratory gas. For the sake of simplicity, only equation 8 continues to be used below, even though equation 8a could be used as well.

Using equation 8, the difference in proportion ΔA_{miG} mentioned at the outset may be written formulaically in a preferred specific embodiment of the method as follows:

$$Δ {A}_{miG} = {}_{in}^{1 Zp} {A}_{miG} - {}_{in}^{2 Zp} {A}_{miG} ≡ ( \frac{1}{( {}^{1 Z p} q + 1 )} \cdot {\sum }_{x = {}^{1 Z p} p - {}^{1 Z p} q}^{{}^{1 Z p} p} {}_{in}^{1 Zp} a {( x )}_{miG}^{tid} ) - ( \frac{1}{( {}^{2 Z p} q + 1 )} \cdot {\sum }_{x = {}^{2 Z p} p - {}^{2 Z p} q}^{{}^{2 Z p} p} {}_{in}^{2 Zp} a {( x )}_{miG}^{tid} ) \qquad ( Eq . 9 )$$

Here, ^{1Zp}p and ^{2Zp}p may differ according to amount as may ^{1Zp}q and ^{2Zp}q. The respective values of p and/or of q may also be the same for both supply phases.

The ascertainment of the functional residual capacity preferably occurs on the basis of a quotient of the corrected difference in amount and the difference in proportion. Using the above notation, the functional residual capacity FRC of a lung of a patient may be represented formulaically as follows:

$$F R C = \frac{{}_{korr}^{2 Zp} Δ {v}_{miG}}{Δ {A}_{miG}} \qquad ( eq . 10 )$$

According to a preferred specific embodiment of the method, the functional residual capacity FRC of a lung of a patient using equations 10, 7a and 9 is:

$$F R C = \frac{{{\sum }^{{n}_{0}}}_{x = {c}_{0}} {}^{2 Zp} Δ {v ( x )}_{m i G}^{t i d} - {}^{i Zp} B}{{}^{1 Zp} {A}_{miG} - {}^{2 Zp} {A}_{miG}} \qquad ( eq . 11 a )$$

or, due to the higher accuracy preferably using equations 10, 7b and 9:

$$F R C = \frac{{{\sum }^{{n}_{0}}}_{x = {c}_{0}} ( {}^{2 Zp} Δ {v ( x )}_{m i G}^{t i d} - {}^{2 Zp} {B}^{tid} ( x ) )}{{}^{1 Zp} {A}_{miG} - {}^{2 Zp} {A}_{miG}} \qquad ( eq . 11 b )$$

where ΔA in the denominator is ascertained in accordance with equation 9.

The present method for ascertaining the FRC is preferably carried out during an artificial respiration of a patient, so that the breaths, during which the above values for calculating the FRC are detected, are breaths for the artificial respiration of the patient.

The artificial respiration of the patient usually continues beyond the end of the ascertainment period or the end of the second supply phase. Preferably, artificial respiration already occurred over multiple breaths, before the presently discussed method for ascertaining an FRC begins. To ensure the ability of the patient's lung to exchange gas, it is therefore preferred, if at the end of an expiration phase of a plurality of breaths of the second supply phase, a respiratory pressure in the airway of the patient and/or in a proximal area of a ventilation line is the PEEP. The PEEP is an overpressure in relation to the ambient pressure, which prevents alveoli of the patient's lung from collapsing at the end of an expiration phase.

While in the related art frequently only values of the expiratory respiratory gas are detected, which are easier to detect in terms of measurement technology, and the likewise required values of the inspiratory respiratory gas are derived from the detected expiratory respiratory gas values, the use of a ventilator, as it was already mentioned above and will be indicated in more detail below, for carrying out the method for the FRC ascertainment, allows for the sensorial detection both of the inspiratory respiratory gas flow as well as of the expiratory respiratory gas flow. The method therefore preferably comprises the sensorial detection both of the inspiratory respiratory gas flow as well as of the expiratory respiratory gas flow. This has the advantage that it is not necessary to use the Haldane transformation normally used among experts for deriving inspiratory respiratory gas values from measured expiratory respiratory gas values. Consequently, it is irrelevant whether the simplifying assumptions at the basis of the Haldane transformation are actually fulfilled or not. Hence, even with the occurrence of the usual interference factors such as moisture, moisture precipitation on sensors in a ventilation line system of the ventilator, the sensorial detection both of the inspiratory as well as of the expiratory respiratory gas flow is able to offer an accuracy advantage in the ascertainment of the FRC compared to the use of the Haldane transformation. On the one hand, it is possible that the prerequisites of the Haldane transformation are not fulfilled. Additionally or alternatively, on the other hand, it is also possible that the aforementioned interfering influences affect the sensorial detection only of the expiratory respiratory gas.

Fundamentally, the metabolically inert gas may be detected directly by a sensor in order to ascertain its proportion in the inspiratory and/or in the expiratory respiratory gas. The metabolically inert as may be a noble gas, such as Helium (He) for example, or it may be a gas that is not or nearly not metabolized by the living organism, such as for example the nitrogen (N₂) that exists in the air anyway, or it may be sulfur hexafluoride (SF₆). The metabolically inert gas is preferably nitrogen, since in that case it is possible to use ambient air as base gas for the inspiratory respiratory gas. To change the proportion of nitrogen in an inspiratory respiratory gas based on ambient air, pure oxygen (O₂) or gas having a higher proportion of oxygen than that of air may be admixed to the inspiratory respiratory gas. Thus, preferably, a gas of the first or the second inspiratory respiratory gas has the natural nitrogen content of the ambient air and the respective other inspiratory respiratory gas has a lower nitrogen content. This ensures that the patient tolerates both the first as well as the second inspiratory respiratory gas. It shall not be precluded, however, that the nitrogen content both in the first as well as in the second inspiratory respiratory gas is lower than in the ambient air, particularly if an illness of an artificially respirated patient requires a higher oxygen content in the inspiratory respiratory gas.

Particularly if nitrogen as a natural component of the ambient air is used as the metabolically inert gas, the nitrogen proportion in the respiratory gas, be it inspiratory or expiratory, may be detected indirectly with sufficient accuracy in that the oxygen content and the carbon dioxide content of the respective respiratory gas are directly sensorially detected. Since it may be assumed with good approximation that a respiratory gas, in particular a respiratory gas based on ambient air, is made up almost solely of nitrogen, oxygen and carbon dioxide, the nitrogen content of the sensorially detected respiratory gas may be determined as the residual content of the respiratory gas that is not oxygen content or carbon dioxide content. Expressed formulaically, this means for the nitrogen content ^{iZp}a(x)_{N}_{₂}^{tid}, as a percentage, in the xth breath of the ith supply phase:

^{iZp}a(x)_{N}_{₂}^{tid}=1−^{iZp}a(x)_{O}_{₂}^{tid}−^{iZp}a(x)_{CO}_{₂}^{tid} (eq. 12)

where ^{iZp}a(x)_{O}_{₂}^{tid} is the oxygen content of the respiratory gas and ^{iZp}a(x)_{CO}_{₂}^{tid} is the carbon dioxide content of the respiratory gas in the same breath.

The denoted proportions as percentage may be volume proportions, mass proportions or molar proportions, depending on which proportion is sensorially detected. Preferably, these are volume proportions in percentage by volume. Likewise, the amounts denoted in the present application may be volumes, masses or molar amounts. The amounts are preferably volumes.

As was already explained several times, the presently presented method is preferably carried out by a ventilator during an artificial respiration of a patient.

The present invention also relates to a ventilator, which is designed both for the at least partial artificial respiration of living patients as well as for carrying out the method as recited in one of the preceding claims, the ventilator comprising:
a first respiratory gas source, which provides a first inspiratory respiratory gas component having a first fraction of a metabolically inert gas,
a second respiratory gas source, which provides a second inspiratory respiratory gas component having a second fraction of the metabolically inert gas differing from the first fraction,
a variably settable mixing device for forming an inspiratory respiratory gas having a variable proportion of metabolically inert gas from the first and/or the second inspiratory respiratory gas component,
a ventilation line system for conveying the inspiratory respiratory gas to a patient-side respiratory gas outlet and for conveying expiratory respiratory gas from a patient-side respiratory gas inlet away from the respiratory gas inlet,
a control valve system, comprising an inspiration valve and an expiration valve,
a pressure changing device for changing at least the inspiratory respiratory gas in the ventilation line system,
a flow sensor system for detecting at least the inspiratory respiratory gas flow,
a gas component sensor system for the indirect or direct detection of the proportion of the metabolically inert gas in the inspiratory and in the expiratory respiratory gas,
a control device, which is designed to control the control valve system and the pressure changing device and which is connected in signal-transmitting fashion to the flow sensor system and to the gas component sensor system for transmitting respective detection signals to the control device.

The first respiratory gas source is preferably a suction port open toward the surroundings, through which ambient air may be aspirated. The first respiratory gas source, however, may also be a storage tank for accommodating first inspiratory respiratory gas or may be a connector coupling for connection to a clinical facility installation for the supply with first inspiratory respiratory gas. In many clinics, supply lines having defined connector counter couplings are permanently installed in the building, the connector counter couplings being accessibly provided for the connector coupling of the ventilator for establishing a connection conducting the first inspiratory respiratory gas.

With reference to the above explanations regarding the method, the second respiratory gas source is preferably an oxygen container, from which oxygen may be supplied into the respiratory gas of the first respiratory gas source. Alternatively or additionally, the second respiratory gas source may also be a connector coupling for connecting to a clinical facility installation. The measure of the supply of gas of the second respiratory gas source into gas of the first respiratory gas source is adjustable at the mixing device. In a simple specific embodiment, it may suffice to switch the mixing device between a blocking state and a defined opening state, no gas being able to flow from the second respiratory gas source into gas from the first respiratory gas source in the blocking state, and an a constant amount of gas flowing per unit of time from the second respiratory gas source into gas of the first respiratory gas source in the defined opening state. Preferably, however, the mixing device may be brought at least by increments or particularly preferably steplessly from the blocking state into different defined opening states, so that at least one inspiratory respiratory gas of the first and the second inspiratory respiratory gas, preferably both inspiratory respiratory gases, are steplessly adaptable to the needs of the respectively respirated patient.

The pressure changing device may be a blower conveying the respiratory gas, at least or only the inspiratory respiratory gas, in the ventilation line system. This applies in particular if ambient air is used as first or second inspiratory respiratory gas or as respiratory gas component.

Additionally or alternatively, however, the pressure changing device may comprise a pressure reducing valve, whose state and thus whose pressure reducing effect is changeable by the control device.

The ventilation line system is used to conduct inspiratory respiratory gas from the first and/or the second respiratory gas source to the patient and to conduct expiratory respiratory gas away from the patient. The respiratory gas outlet and the respiratory gas inlet may be one and the same formation, for example the proximal opening of an endotrachealtubus. However, they may also be different openings.

The ventilation line system may have physically separate sections for inspiratory and for expiratory respiratory gas. The sections may be merged by a so-called Y connector into a common line section that is used both for expiratory as well as for inspiratory respiratory gas. The line section used both for expiratory as well as for inspiratory respiratory gas is preferably a line section, which is situated between the Y connector and the patient, that is, which extends in particular to the respiratory gas inlet and to the respiratory gas outlet.

The flow sensor system may be any sensor system for detecting at least the inspiratory respiratory gas flow, preferably also the expiratory respiratory gas flow, such as for example a hot wire anemometer. The flow sensor system preferably comprises a pressure difference flow sensor system having a variable flow resistor and having on both sides of the flow resistor—when viewed along the flow path of the respiratory gas—detection points provided for detecting the pressure of the respiratory gas. Using the flow sensor system, it is thus possible to determine, not only the respiratory gas flow, but at the same time also the respiratory gas pressure. So that the flow sensor system is able to detect both the flow and, if indicated, the pressure of the inspiratory as well as of the expiratory respiratory gas, it is preferably situated in the aforementioned jointly used line section between the Y connector and the respiratory gas inlet or the respiratory gas outlet.

The development of the ventilator for carrying out the method for ascertaining the FRC described and refined further above is implemented with regard to the required control interventions and with regard to the required data processing by a corresponding development of the control device. The control device is furthermore developed to control components of the ventilator in such a way that the method steps defined further above are carried out in the ventilator.

Via the possibility of changing the operating state of at least the pressure changing device and the control valve system, the control device also receives signals from the mentioned sensors, from which the control device is able to ascertain a respiratory gas flow and a proportion of the metabolically inert gas in the respiratory gas. This applies at least with respect to the inspiratory or the expiratory respiratory gas, preferably with respect to the inspiratory and the expiratory respiratory gas.

The control device furthermore makes use of a time signal, provided via a time measuring device integrated in the control device or connected to the control device, which allows the control device to ascertain from an ascertained gas flow an amount of gas that flowed over a period of time.

With reference to the sensorial detection of the respiratory gas components explained above with respect to the method, the gas component sensor system preferably comprises at least one of the following sensors:
an oxygen sensor for detecting an oxygen content in the inspiratory and in the expiratory respiratory gas, and
a carbon dioxide sensor for detecting a carbon dioxide content in the inspiratory and in the expiratory respiratory gas.

The gas component sensor system preferably comprises both an oxygen sensor as well as a carbon dioxide sensor. In order to facilitate operation and to detect oxygen and carbon dioxide in the respiratory gas as synchronously as possible, the oxygen sensor and the carbon dioxide sensor are preferably accommodated in a common housing. The carbon dioxide sensor may be a non-dispersive infrared sensor. The oxygen sensor may be an oxygen sensor operating according to the principle of luminescence quenching.

Since the mentioned sensors often detect only a physical quantity representing the partial pressure of the gas detectable by the respective sensor, the ventilator preferably also comprises a barometer for detecting the ambient air pressure, in order to be able to infer a proportion of the detected gas in the respiratory gas as a whole from the detected quantities, which represent a partial pressure. Thus, the average tidal proportion ^{iZp}a(n)_{Gas}^{tid} of a gas in the respiratory gas during the nth breath in the ith supply phase is:

$${}^{iZp} a {( n )}_{Gas}^{tid} = \frac{{\int }_{t {=}_{{in}^{{t}_{0}}}}^{e {x}^{t} e n d} \vert \dot{V} \vert \cdot \frac{{}^{iZp} {p}_{Gas}^{tid} ( t )}{{}^{iZp} {p}_{amb}^{tid} ( t ) + {}^{iZp} {p}_{awy}^{tid} ( t )} d t}{{\int }_{t {=}_{{in}^{{t}_{0}}}}^{e {x}^{t} e n d} \vert \dot{V} \vert \cdot d t} \qquad ( eq . 13 )$$

with |\dot{V}| as the amount of the respiratory gas flow detected by the flow sensor system during the nth breath, with ᵢₙt₀ as the time of the start of the inspiration phase and with ₑₓt_{end} as the time of the end of the expiration phase of the nth breath in the ith supply phase, and with ^{iZp}p_{Gas}^{tid}(t) as the partial pressure, sensorially detected during the nth breath, of the gas detectable by the respective sensor, ^{iZp}p_{amb}^{tid}(t) as the ambient pressure detected during the nth breath and with ^{iZp}p_{awy}^{tid}(t) as the overpressure or underpressure sensorially detected during the nth breath preferably by the flow sensor system and/or by a separate pressure sensor, in particular in a proximal section of the ventilation line system. The airway overpressure or underpressure is preferably detected in a section of the ventilation line system through which both expiratory as well as inspiratory respiratory gases flow.

The control device is designed to carry out the computation of equation 13 on the basis of the indicated sensor detection values.

In the related art it is often necessary for determining an FRC to divert respiratory gas from the respiratory gas flow in the ventilation line system and to detect and process it by measurement technology in a separate measurement branch. Such a detection of respiratory gas by measurement technology in a so-called bypass flow increases the risk of inaccuracies in determining the FRC. The detection of respiratory gas by measurement technology in a bypass flow primarily presents the problem of the sufficient synchronization of the measurement results obtained in the bypass flow with the ventilation process occurring in the main flow path. The respiratory gas diverted for processing by measurement technology into a bypass flow section physically separated from a main flow section for supplying the patient provides measured values by the sensorial detection at a point in time, which may be offset from the point in time at which the sensorially detected respiratory gas is supplied to the lung or flows out of the latter. However, since according to the above representation tidal quantities, that is, quantities specific to an individual breath, are used for ascertaining the functional residual capacity, it is necessary to be able to assign the measured values obtained in the bypass section unequivocally to a breath. Since the method described above may be carried out in the presently discussed ventilator, this synchronization problem does not exist in the ventilator, for the ventilator allows for a sensorial detection in a main flow section. A main flow section in the sense of the present invention is a section of the ventilation line system, whose flowing respiratory gas, which is conducted by it and which is preferably both the inspiratory as well as the expiratory respiratory gas, is directly fed to the patient-side respiratory gas outlet at at least 95 vol %, preferably at at least 98 vol %, particularly preferably, neglecting possible leakages, at 100 vol %, or was directly discharged from the lung of the patient via the patient-side respiratory gas inlet into the ventilation line system. It is therefore preferably provided for the gas component sensor system to be situated in a main flow section of the ventilation line system for detecting the proportion of the metabolically inert gas in the inspiratory and in the expiratory respiratory gas, through which both the inspiratory respiratory gas fed to the patient as well as the expiratory respiratory gas flowing away from the patient flow.

In principle, it is conceivable to operate the mixing device manually in order to end the first supply phase and to start the second supply phase. Preferably, however, the control device is designed to control the mixing device so as to change the proportion of metabolically inert gas in the inspiratory respiratory gas by controlling the mixing device. The mixing device may comprise an actuator, which is controllable by the control device. The mixing device may comprise a valve, whose degree of opening is changeable by the control device.

The present invention is explained in greater detail below with reference to the attached drawings. The figures show:

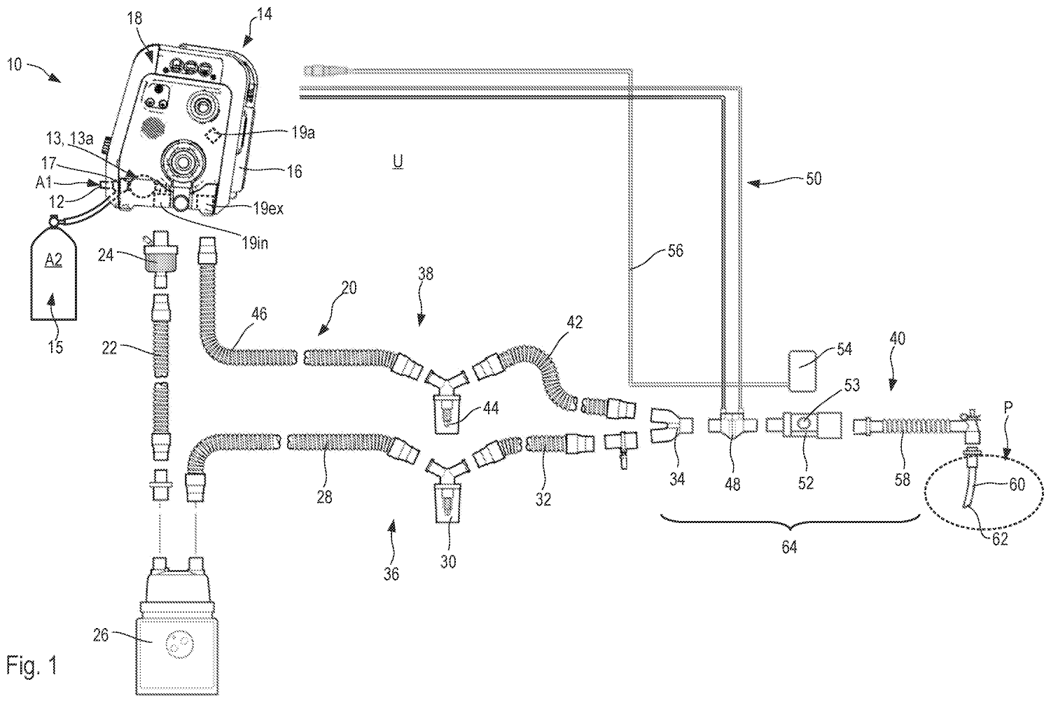

FIG. 1 a rough schematic view of a ventilator according to the invention,

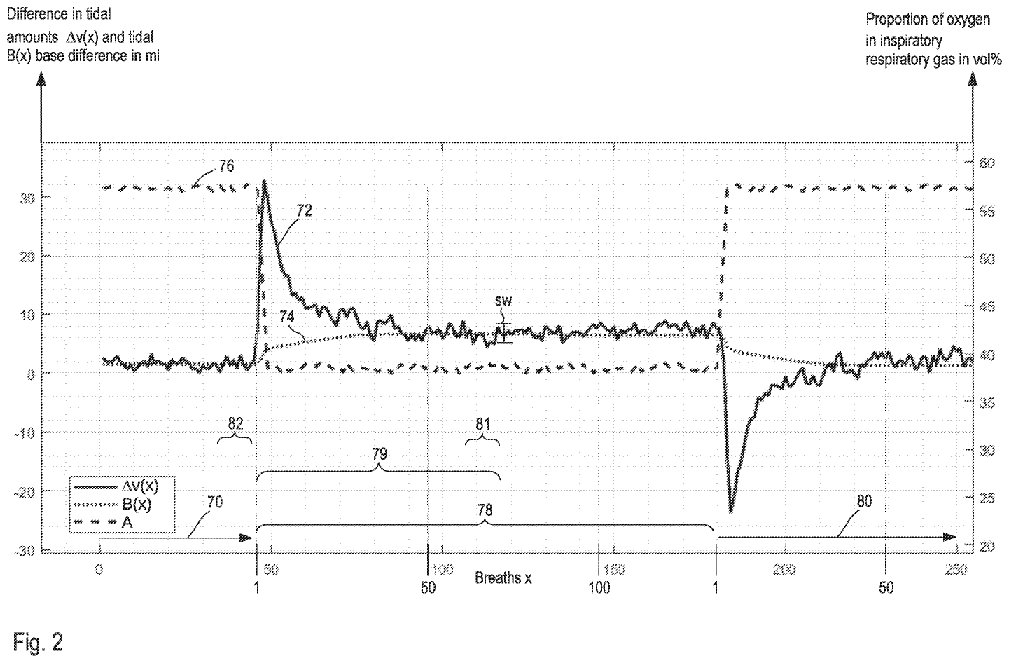

FIG. 2 a graphic representation of the characteristic curve of a difference in tidal amounts, of a proportion of nitrogen in the inspiratory respiratory gas and of a difference in base amount during a second supply phase, including a representation of the end of a preceding first supply phase and a subsequent third supply phase, and

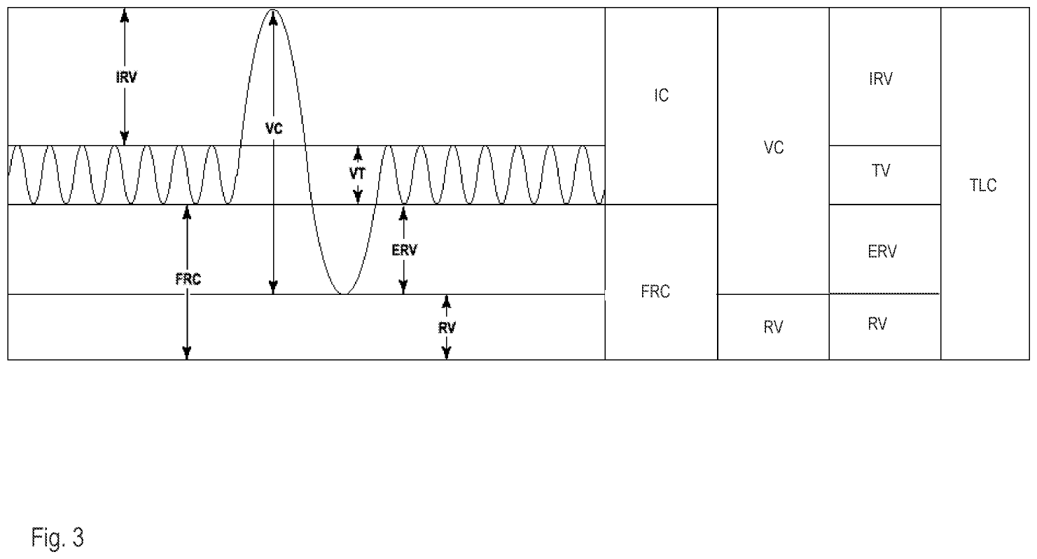

FIG. 3 a representation of respiratorily relevant breath or lung volumes.

In FIG. 1, a specific embodiment of a ventilator according to the invention is generally denoted by reference numeral 10. Ventilator 10 comprises a first respiratory gas source 12 in the form of a suction port opening toward surroundings U of ventilator 10. An output-variable blower 13, which is controllable by a control device 14, allows for ambient air to be aspirated as the first respiratory gas component A1. Blower 13 and control device 14 are accommodated in the same housing 16. This housing also accommodates valves known per se, such as an inspiration valve 19in and an expiration valve 19ex. Control device 14 additionally comprises a time measuring device 19a.

Furthermore, a second respiratory gas source 15 is connected to housing 16 in a flow-connecting manner. The second respiratory gas source 15 may be a pressurized gas cylinder, for instance with pressurized pure oxygen stored therein as a second respiratory gas component A2.

The first respiratory gas component A1 aspirated at the first respiratory gas source 12 and the second respiratory gas component A2 supplied by the second respiratory gas source 15 are conducted to a mixing valve 17, which mixes, as a function of its position, preferably steplessly, the two respiratory gas components into an inspiratory respiratory gas having an arbitrary mixture ratio from 100 vol % of the first respiratory gas component A1 and 0 vol % of the second respiratory gas component A2 to 0 vol % of the first respiratory gas component A1 and 100 vol % of the second respiratory gas component A2. The mixing valve 17 and thus the mixture ratio of the inspiratory respiratory gas is likewise controllable or adjustable by control device 14.

In the illustrated example, N₂ is used as the metabolically inert gas. Since the first respiratory gas component A1 has an N₂ fraction of approximately 71 vol % and the second respiratory gas component has an N₂ fraction of approximately 0 vol %, the inspiratory respiratory gas mixed by mixing valve 17 may have an N₂ proportion of between 0 and 71 vol %. Such an inspiratory respiratory gas is breathable by any land-dwelling creature of this planet that would be a candidate for artificial respiration. The change of the mixture ratio of the respiratory gas components may preferably be switched temporally within one breath, particularly preferably with an expiration from a first, earlier mixture ratio to a second, later mixture ratio.

The control device 14 of ventilator 10 has an input/output device 18, which comprises numerous switches such as push-button switches and rotary switches, in order to be able, if necessary, to input data into control device 14. Blower 13 of first respiratory gas source 12 may be changed in its conveying capacity by the control device, in order to change the amount of respiratory gas that is conveyed per unit of time. Blower 13 is therefore in the present exemplary embodiment a pressure changing device 13a of ventilator 10.

A ventilation line system 20, comprising five flexible hoses in the present example, is connected to the line leading away from blower 13 and toward patient P with the inspiration valve 19in situated in between. A first inspiratory ventilation hose 22 runs from a filter 24 situated between it and inspiration valve 19in to an optional conditioning device 26, where the respiratory gas supplied by the respiratory gas source 12 is humidified to a specified degree of humidity and, if indicated, is provided with aerosol medications. Filter 24 filters and cleans the ambient air supplied by blower 13.

A second inspiratory ventilation hose 28 leads from the optional conditioning device 26 to an inspiratory water trap 30. A third inspiratory ventilation hose 32 leads from water trap 30 to a Y connector 34, which connects the distal inspiration line 36 and the distal expiration line 38 to a combined proximal inspiratory-expiratory ventilation line 40.

A first expiratory ventilation hose 42 runs from the Y connector 34 back to housing 16 to an expiratory water trap 44 and from there a second expiratory ventilation hose 46 runs to housing 16, where the expiratory respiratory gas is discharged into the surroundings via expiration valve 19ex.

On the combined inspiratory-expiratory side of Y connector 34 near the patient, the Y connector 34 is directly followed by a flow sensor 48, here: a differential pressure flow sensor 48, which detects the inspiratory and the expiratory flows of respiratory gas toward patient P and away from patient P. A line system 50 transmits the gas pressure prevailing on both sides of a variable flow obstruction, known per se, in flow sensor 48 to control device 14, which calculates from the transmitted gas pressures and in particular from the difference of the gas pressures the amount of inspiratory and expiratory respiratory gas flowing per unit of time.

In the direction away from Y connector 34 and toward patient P, flow sensor 48 is followed by a measuring cuvette 52 both for the non-dispersive infrared detection of a predetermined volumic gas proportion in the respiratory gas, here carbon dioxide (CO₂) by way of example, as well as for the luminescence-based detection of the volumic gas proportion of oxygen (O₂). The CO₂ proportions and the O₂ proportions are of interest both in the inspiratory respiratory gas as well as in the expiratory respiratory gas, since the change of the CO₂ proportion and of the O₂ proportion between the inspiration and the expiration is a measure of the metabolic ability of the patient's lung. FIG. 1 shows one of the lateral windows 53, through which infrared light may be radiated into the measuring cuvette 52 or may radiate out of the latter, depending on the orientation of a combined CO₂—O₂ gas sensor 54 that is releasably coupled to the measuring cuvette.

Gas sensor 54 may be coupled to measuring cuvette 52 in such a way that the gas sensor 54 is able both to radiate infrared light through the measuring cuvette 52 as well as to excite a luminophore-containing measuring surface of the measuring cuvette 52 to radiate.

From the intensity of the infrared light, more precisely from its spectral intensity, it is possible to infer, in a manner known per se, the amount or the proportion of a predetermined gas in the respiratory gas flowing through the measuring cuvette 52. The predetermined gas, here: CO₂, absorbs infrared light of a defined wavelength. The intensity of the infrared light of this wavelength following the passage depends essentially on the absorption of the infrared light of this wave length by the predetermined gas. A comparison of the intensity of the infrared light of the defined wavelength with a wavelength of the infrared light, which does not belong to an absorption spectrum of an expected gas proportion in the respiratory gas, provides information about the proportion of the predetermined gas in the respiratory gas.

From the radiation response of the luminophore-containing measuring surface of measuring cuvette 52 to the above-described excitation by gas sensor 54, which is detected by gas sensor 54, it is possible to ascertain the volumic O₂ proportion in the respiratory gas by taking into account an intensity difference and/or a phase difference between the preferably modulated excitation radiation and the excited radiation. 02 acts as quencher substance for the luminophore of the measuring surface and decisively influences the response radiation with respect to intensity and/or phase shift.

Gas sensor 54 is therefore connected to the control device 14 of ventilator 10 via a data line 56 and transmits the described intensity information via the data line 56 to control device 14.

In the direction toward patient P, measuring cuvette 52 is followed by a further hose section 58, on which an endotrachealtubus 60 is attached as the respiratory interface to patient P. A proximal opening 62 of endotrachealtubus 60 is both a respiratory gas outlet opening, through which inspiratory respiratory gas is fed through endotrachealtubus 60 into patient P, as well as a respiratory gas inlet opening, through which expiratory respiratory gas is conducted out of the patient and back into endotrachealtubus 60.

The entire ventilation line system is a main flow line, without branching of a bypass flow line. The proximal single strand section of the Y connector 34, flow sensor 48, measuring cuvette 52 and hose section 58 form a main flow section 64 situated outside of the body of patient P, through which both inspiratory as well as expiratory respiratory gases flow.

Control device 14 is designed to control blower 13 and mixing device 17 according to the method described at the outset, in order to ascertain from the detection values, which are detected by gas sensor 54 and flow sensor 48, a functional residual capacity FRC of the lung of patient P.

For this purpose, initially, in a first supply phase 70, a first inspiratory respiratory gas is fed to patient P, which is formed from a mixture of the two respiratory gas components A1 and A2, so that the inspiratory respiratory gas has a higher oxygen content than first respiratory gas component A1, that is, the ambient air, by itself.

The end of this first supply phase 70 is indicated in the diagram of FIG. 2 by an arrow.

FIG. 2 shows three diagrams and has two scales for this purpose. The left ordinate scale in FIG. 2 refers to differences in tidal amounts in milliliters and applies to the difference in tidal amounts ^{iZp}Δv(n)_{N}_{₂}^{tid}, abbreviated in FIG. 2 as Δv(x), represented by a solid line and labeled by reference numeral 72, as it is determined for each breath by equation 1 from the values detected by gas sensor 54. It also applies to the tidal base difference ^{iZp}B^{tid}, abbreviated in FIG. 2 as B(x), represented by a dotted line and labeled by reference numeral 74, as it is ascertained in accordance with equation 6.

The right ordinate scale in FIG. 2 refers to the proportion ᵢₙ^{iZp}A_{O}_{₂}, abbreviated in FIG. 2 as A, of oxygen in the inspiratory respiratory gas in percentage by volume. The graph of the oxygen proportion in the inspiratory respiratory gas is shown in FIG. 2 by a dashed line and labeled by reference numeral 76.

The abscissa of the representation of FIG. 2 indicates the breaths x. The abscissa has two scales. one of which starts at zero and increments by the value 1 for each breath in the considered period. The other scale increments likewise by the value 1, but starts to count in each supply phase anew with the value 1.

Since in the first supply phase 70, due to the greater admixture of the second respiratory gas component A2, this first inspiratory respiratory gas contains a smaller amount or a smaller proportional amount of nitrogen as the metabolically inert gas than the second inspiratory respiratory gas, the first supply phase 70 corresponds to a wash-out phase described in the introduction of the specification.

In the process, the composition of the first inspiratory respiratory gas and of the first expiratory respiratory gas formed from it is detected tidally, that is, for every breath. From the flow information obtained from flow sensor 48 as respiratory gas volume flowing inspiratorily and expiratorily per unit of time, and from the volume proportions of oxygen and carbon dioxide both of the inspiratory respiratory gas as well as of the expiratory respiratory gas obtained from gas sensor 54, it is possible to obtain for every breath both the inspiratorily administered amounts as well as the expiratorily discharged amounts of oxygen, carbon dioxide and nitrogen on the simplifying, but sufficiently accurate assumption that the inspiratory and the expiratory respiratory gas contains no further components of a significant amount beyond oxygen, carbon dioxide and nitrogen.

Thus, it is possible to ascertain the differences in tidal amounts according to equation 1 directly from the detection results available to control device 14. Together with the differences in tidal amounts, it is also possible for control device 14 to ascertain their moving arithmetic average according to equation 2. In the same way, the average proportion of nitrogen in the first inspiratory respiratory gas is ascertained in accordance with equation 8 or 8a. The ascertained values are stored in a data storage unit of control device 14.

If the moving average of the differential value according to equation 2 for the first supply phase 70 lies below a predetermined threshold value according to amount or is equal to the same, then control device 14 ends the first supply phase by adjusting the mixing valve as mixing device 17 and feeds a second inspiratory respiratory gas to patient P, whose nitrogen component is changed with respect to the first inspiratory respiratory gas, that is, increased in the present example. The second supply phase 78 thus starts, as may be seen in FIG. 2 by the left value 1 in the lower abscissa scale. The second supply phase 78 continues for about 130 breaths.

The amount of respiratory gas component A2 that is admixed to respiratory gas component A1 is lower in the second supply phase 78 than in the first supply phase 70. Due to the adjustment of mixing valve 17, the oxygen proportion in the respiratory gas falls abruptly from approximately 57 vol % to approximately 38 vol %. The oxygen proportion, however, is still higher than in pure ambient air.

The ascertainment period over which the FRC is ascertained begins with the second supply phase 78. It is not necessary for the FRC to be ascertained in real time during the second supply phase, but rather it is only necessary that the differences in tidal amounts used for ascertaining the FRC originate from the ascertainment period.

From the start of the second supply phase, a difference in tidal amounts is therefore calculated for every breath in accordance with equation 1. If a sufficient number of breaths for averaging have already occurred, the moving average of the differences in tidal amounts is also formed in accordance with equation 2. Again, if the moving differential value according to equation 2 falls to or below a threshold value predetermined for this purpose, the ascertainment period ends.

The characteristic curve of the difference in tidal amounts over the observed period is indicated by graph 72. Since, due to dead volumes in the lung of the patient, there is still respiratory gas of the first supply phase with a lower nitrogen proportion in the patient's lung, patient P initially exhales second expiratory respiratory gas beginning with the start of the second supply phase 78, which has a higher volumic proportion of nitrogen than the second inspiratory respiratory gas. The difference in tidal amounts for the breaths at the start of the second supply phase 78 is therefore positive and deviates significantly, on the one hand, from the value 0, which is reached when the expiratory and the inspiratory respiratory gas have a nitrogen proportion of identical magnitude. The difference in tidal amounts 72, on the other hand, also deviates significantly from base difference 74 toward the end of first supply phase 70. So that nitrogen is washed out of the patient's lung in the second supply phase 78, the difference in tidal amounts 72 falls with increasing distance from the start of the second supply phase 78, until it levels off around a constant offset value starting approximately with the 50th breath of the second supply phase 78. Starting approximately with this 50th breath of the second supply phase 78, the difference in tidal amounts 72 no longer changes substantially, but is henceforth essentially only influenced by interference effects of ventilator 10, such as leakages and the like.

As already mentioned above, the tidal base difference 74 is ascertained in accordance with the above equation 6. Starting from the value of the tidal base difference 74 toward the end of the first supply phase 70, it initially rises sharply, then ever more slightly, until it essentially converges to the offset value of the difference in tidal amounts 72.

Following the end of the ascertainment period, the proportion of nitrogen in the second inspiratory respiratory gas is ascertained in accordance with equation 8 or 8a. When working with equation 8a, the constant p should be chosen to be greater than 100 and the constant q should be chosen to be no greater than 50, so that the detection values used for the application of equation 8a originate from the range, for example the end detection range 81 in the second supply phase 78, in which the difference in tidal amounts 72 has an essentially constant value or a value that oscillates around a constant value.

The FRC is then preferably calculated from equation 11b, in order to obtain the FRC with high accuracy. Alternatively, however, equation 11a could be used as well.

Since the base difference is ascertainable at the end of the first supply phase 70, before the second supply phase 78 begins, and since the nitrogen proportion in the inspiratory respiratory gas of the second supply phase 78 is known at least as a setpoint value, by using the setpoint value for the nitrogen proportion of the second supply phase 78, it is possible to ascertain the FRC even in real time during an artificial respiration of patient P. For then all data required for calculating equation 11b are known at the time of every breath of the second supply phase.

Although the entire second supply phase 78 may be used for ascertaining the FRC, a shorter ascertainment period 79 suffices. Preferably, it suffices if the ascertainment period 79 begins together with the second supply phase 78 and if it ends in the range in which the difference in tidal amounts 72 and the tidal base difference do not differ according to amount by more than a predetermined small threshold value sw.

FIG. 2 shows the start of a third supply phase 80, with which again first respiratory gas having a higher oxygen proportion is fed to patient P. Consequently, nitrogen still present due to the dead spaces of the lung is washed out of the patient's lung, so that the expiratory respiratory gas has a higher proportion of nitrogen than the inspiratory respiratory gas of the same breath. The difference in tidal amounts is thus negative and diminishes in with increasing distance from the start of the third supply phase 80. Since in the first and third supply phases 70 and 80, respectively, the same first inspiratory respiratory gas is used, the same conditions set in with the continuance of the third supply phase 80 as toward the end of the first supply phase 70.

Alternatively, the FRC may also be calculated in accordance with equation 11a, instead of equation 11b, the base difference B being calculated for this purpose as the average value of the difference in tidal amounts Δv(x) over an end detection segment 81 in second supply phase 78 or, although less preferred due to the lower achievable accuracy of the FRC ascertainment, over a start detection segment 82 in first supply phase 70. The end detection segment 81 is closer to the end of the detection period 79 than to its start or to the simultaneous start of second supply phase 78. The start detection segment 82 is closer to the start of the second supply phase 78 than to the start of first supply phase 70.

FIG. 3, which originates from the source “Vihsadas” in en.wikipedia, explains the various partial volumes of a patient's lung in order to clarify what precisely is meant by a functional residual capacity according to the present invention.

In FIG. 3 on the left, a spirometric curve of a respiratory gas volume in a patient's lung is plotted over multiple breaths.

A total lung capacity TLC is the volume that is theoretically feedable into a lung starting from a completely collapsed lung of a patient to the maximally possible inhalation. This value is purely theoretical, since a completely collapsed lung would be lethal for patient P.

In a functioning lung of a patient, a residual volume RV therefore always remains, which the patient P is not able to drive out of his lung even with the greatest effort. The vital capacity VC of the lung of a patient is the volume, which the patient P is able to supply to his lung or remove from his lung between a state maximally exhaled with maximum effort and a state maximally inhaled with maximum effort.

In normal breathing, occurring essentially without effort, the tidal volume TV is fed to the lung of the patient P and is again removed from the latter. If patient P, starting from an effortlessly exhaled state, inhales maximally, then he supplies to his lung the so-called inspiration capacity IC of respiratory gas. If, starting from an effortlessly inhaled state, he inhales maximally by mobilizing his entire inspiratory force, then he fills his lung additionally with the inspiratory reserve volume, the IRV. If, starting from an effortlessly exhaled state, he exhales maximally by mobilizing his entire expiratory force, patient P thereby exhales the expiratory reserve volume ERV of his lung.

The sum of the residual volume RV and the vital capacity VC corresponds to the total lung capacity TLC just as the sum of the residual volume RV, the expiratory reserve volume ERV and the inspiratory capacity IC.

The difference between the total lung capacity TLC and the inspiratory capacity IC is the functional residual capacity of the lung of the patient. The latter also results from the sum of the residual volume and the expiratory reserve volume ERV. The functional residual capacity FRC is equally the total lung capacity TLC minus the tidal volume and further minus the inspiratory reserve volume IRV.

1. A method for ascertaining a functional residual capacity of a lung of a patient, comprising the following steps:
supplying a first inspiratory respiratory gas having a first proportion of a metabolically inert gas during a first temporal supply phase,
following the first supply phase: supplying a second inspiratory respiratory gas, differing from the first and having a second proportion of the metabolically inert gas differing from the first, during a second temporal supply phase,
ascertaining a difference in amount occurring during the second supply phase, which represents a difference amount for an ascertainment period between an amount of inspiratory metabolically inert gas and an amount of expiratory metabolically inert gas, the ascertainment period not ending after the second supply phase,
ascertaining the functional residual capacity by taking into account the difference in amount and a difference in proportion between a first proportion quantity, which represents the first proportion of the metabolically inert gas in the first inspiratory working gas, and a second proportion quantity, which represents the second proportion of the metabolically inert gas in the second inspiratory working gas, and
ascertaining a base difference, which represents a difference between a tidal amount of inspiratory metabolically inert gas and a tidal amount of expiratory metabolically inert gas in at least one of the first and the second supply phase;
wherein the ascertainment of the functional residual capacity occurring on the basis of a corrected difference in amount and the difference in proportion, the corrected difference in amount being formed by taking into account the base difference when ascertaining the difference in amount.

2. The method as recited in claim 1,
the base difference comprises at least one average value from a plurality of differences in tidal amounts between respectively a tidal amount of inspiratory metabolically inert gas and a tidal amount of expiratory metabolically inert gas for a plurality of breaths in at least one of the first and the second supply phase.

3. The method as recited in claim 2,
wherein at least one of the base difference comprises an average value from a plurality of differences in tidal amounts between respectively a tidal amount of inspiratory metabolically inert gas and a tidal amount of expiratory metabolically inert gas for a plurality of breaths in a temporal start detection segment in the first supply phase, the start detection segment being closer to the start of the second supply phase than to the start of the first supply phase,
and
the base difference comprises an average value from a plurality of differences in tidal amounts between respectively a tidal amount of inspiratory metabolically inert gas and a tidal amount of expiratory metabolically inert gas for a plurality of breaths in a temporal end detection segment in the second supply phase, the end detection segment being closer to the end of the second supply phase than to its start.

4. The method as recited in claim 1,
wherein at least one of the base difference comprises at least during a segment in the second supply phase and in the detection period a tidal base difference, which is determined for a breath depending on a proportion of the metabolically inert gas in the respiratory gas of the respective breath.

5. The method as recited in claim 1,
wherein the corrected difference in amount corresponds to a sum of corrected differences in tidal amounts over a number of breaths in the ascertainment period, a corrected difference in the tidal amounts being formed for every breath from a difference of a difference in the tidal amounts of this breath and a base difference associated with the breath, the difference in the tidal amounts being formed for every breath by the difference between a tidal amount of inspiratory metabolically inert gas and a tidal amount of inspiratory metabolically inert gas of this breath.

6. The method as recited in claim 1,
wherein at least one of the first proportion quantity comprises or is an average value, formed over a plurality of breaths in the first supply phase, of the first proportion of the metabolically inert gas in the first inspiratory or expiratory working gas, and
the second proportion quantity comprises or is an average value, formed over a plurality of breaths in the second supply phase, of the second proportion of the metabolically inert gas in the second inspiratory or expiratory working gas.

7. The method as recited in claim 6,
wherein at least one of the plurality of breaths, over which the first proportion quantity is ascertained as an average value, is closer to the start of at least one of the ascertainment period and of the second supply phase than to the start of the first supply phase,
and
the plurality of breaths, over which the second proportion quantity is ascertained as an average value, is closer to at least one of the end of the ascertainment period and of the second supply phase than to the start of the ascertainment period or the second supply phase.

8. The method as recited in claim 1,
wherein the ascertainment of the functional residual capacity occurs on the basis of a quotient of the corrected difference in amount and the difference in proportion.

9. The method as recited in claim 1, carried out at a ventilator,
wherein at the end of a plurality of breaths during the second supply phase at the end of an expiration phase, a respiratory pressure in at least one the airway of of the patient and in a proximal area of a ventilation line is the PEEP.

10. The method as recited in claim 1,
further comprising a sensorial detection both of an inspiratory respiratory gas flow as well as of an expiratory respiratory gas flow.

11. The method as recited in claim 1,
carried out by a ventilator during an artificial respiration of a patient.

12. A ventilator, which is designed both for the at least partial artificial respiration of living patients as well as for carrying out the method as recited in one of the preceding claims, the ventilator comprising:
a first respiratory gas source, which provides a first inspiratory respiratory gas component having a first fraction of a metabolically inert gas,
a second respiratory gas source, which provides a second inspiratory respiratory gas component having a second fraction of the metabolically inert gas differing from the first fraction,
a variably settable mixing device for forming an inspiratory respiratory gas having a variable proportion of metabolically inert gas from at least one of the first and the second inspiratory respiratory gas component,
a ventilation line system for conveying the inspiratory respiratory gas to a patient-side respiratory gas outlet and for conveying expiratory respiratory gas away from a patient-side respiratory gas inlet,
a control valve system, comprising an inspiration valve and an expiration valve,
a pressure changing device for changing at least the inspiratory respiratory gas in the ventilation line system,
a flow sensor system for detecting at least the inspiratory respiratory gas flow,
a gas component sensor system for the indirect or direct detection of the proportion of the metabolically inert gas in the inspiratory and in the expiratory respiratory gas,
a control device, which is designed to control the control valve system and the pressure changing device and which is connected in signal-transmitting fashion to the flow sensor system and to the gas component sensor system for transmitting respective detection signals to the control device.

13. The ventilator as recited in claim 12,
wherein the gas component sensor system comprises at least one of the following sensors:
an oxygen sensor for detecting an oxygen content in the inspiratory and in the expiratory respiratory gas, and
a carbon dioxide sensor for detecting a carbon dioxide content in the inspiratory and in the expiratory respiratory gas.

14. The ventilator as recited in claim 12,
wherein the gas component sensor system is situated in a main flow section of the ventilation line system for detecting the proportion of the metabolically inert gas in the inspiratory and in the expiratory respiratory gas, through which both the inspiratory respiratory gas fed to the patient as well as the expiratory respiratory gas flowing away from the patient flow.

15. The ventilator as recited in claim 14,
wherein the main flow section conducts at least 95 vol % of the inspiratory and of the expiratory respiratory gas.

16. The ventilator as recited in claim 12,
wherein the control device is designed for controlling the mixing device so as to change the proportion of metabolically inert gas in the inspiratory respiratory gas by controlling the mixing device.

17. The ventilator as recited in claim 13,
wherein the gas component sensor system is situated in a main flow section of the ventilation line system for detecting the proportion of the metabolically inert gas in the inspiratory and in the expiratory respiratory gas, through which both the inspiratory respiratory gas fed to the patient as well as the expiratory respiratory gas flowing away from the patient flow.

18. The ventilator as recited in claim 17,
wherein the main flow section conducts at least 95 vol % of the inspiratory and of the expiratory respiratory gas.

19. The ventilator as recited in claim 13,
wherein the control device is designed for controlling the mixing device so as to change the proportion of metabolically inert gas in the inspiratory respiratory gas by controlling the mixing device.

20. The ventilator as recited in claim 14,
wherein the control device is designed for controlling the mixing device so as to change the proportion of metabolically inert gas in the inspiratory respiratory gas by controlling the mixing device.
